(19) United States
(12) Patent Application Publication
(10) Pub. No.: US 2008/0311194 A1
(43) Pub. Date: Dec. 18, 2008

(54) Dispersible Tablets Comprising Deferasirox

(76) Inventors: Florian Battung, Paris (FR); Jean-Pierre Cassiere, Rueil Malmaison (FR)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/090,326

(22) PCT Filed: Oct. 17, 2006
(86) PCT No.: PCT/EP2006/010020

§ 371 Date: Apr. 15, 2008

(30) Foreign Application Priority Data
Oct. 19, 2005 (FR) 0553182

(51) Int. Cl. A61K 9/20; A61K 31/4196; A61P 43/00

(52) U.S. Cl. 424/465; 514/383

(57) ABSTRACT

The present invention pertains to a dispersible tablet comprising (a) Compound I of the formula

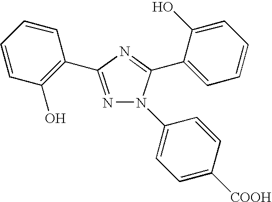

or a pharmaceutically acceptable salt thereof present in an amount of from 42% to 65% by weight based on the total weight of the tablet and (b) at least one pharmaceutically acceptable excipient suitable for the preparation of dispersible tablets and to process for making said dispersible tablet.

The present invention relates to dispersible tablets, e.g. pharmaceutical dispersible tablets, comprising 4-[3,5-bis(2-hydroxyphenyl)-[1,2,4]triazol-1-yl]benzoic acid or a pharmaceutically acceptable salt thereof, and is hereinafter referred to as Compound I.

Compound I is an orally active iron chelator that is indicated in the treatment of iron overload in transfusion dependent anemias, in particular thalassemia major, thalassemia intermediate and in sickle cell disease to reduce iron-related morbidity and mortality. Compound I can also be used in the treatment of hemochromatosis.

Clinical thalassemia (major and intermedia) are hereditary disorders characterized by defective production of hemoglobin, which leads to decreased production and increased destruction of red blood cells.

Sickle cell disease is caused by a mutation in the hemoglobin-Beta gene leading to the production of abnormal hemoglobin S. Normal red blood cells die after 120 days and sickle cells (red blood cells with hemoglobin S) are destroyed more rapidly (10 to 20 days) causing anemia. This anemia is what gives the disease its commonly known name—sickle cell anemia.

Hemochromatosis, the most common form of iron overload disease, is an inherited disorder that causes the body to absorb and store too much iron. The extra iron builds up in organs and damages them. Without treatment, the disease can cause these organs to fail.

Patients with sickle cell disease or thalassemia, who receive significant numbers of blood transfusions and patients with hemochromatosis require therapy to remove iron from the body, called chelation therapy.

Compound I has the following formula:

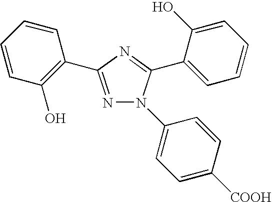

Compound I in the free acid form, salts thereof and its crystalline forms are disclosed in the International Patent Publication WO 97/49395, which is hereby incorporated by reference.

Typically, prescribed daily dosages of Compound I for the treatment of thalassemia are high, e.g. 5 to 40 mg/kg of body weight/day in adults or children. In children, the dosage is preferably 5 to 30 mg/kg of body weight/day. Depending on age, individual condition, mode of administration, and the clinical picture in question, effective daily dosing, e.g. 350 to 2800 mg of Compound I, are administered to patients of 70 kg body weight. Due to the high daily dosing, the patient may have to take 6 tablets or more per day. Thus, there is a need for an oral dosage form allowing the patient to take a reduced number of tablets, that is convenient to administer to patients and that provides a pharmacologically active daily dosage amount of Compound I.

Compound I does not have good compression properties. It is a problem to prepare dispersible tablets with high drug load when the drug does not have appropriate compression properties. Present inventors have now surprisingly found that Compound I may be formulated in form of a dispersible tablet having a drug load of about 1000 mg of Compound I and which is convenient to administer and stable.

By “dispersible tablet” is meant a tablet which disperses in aqueous phase, e.g. in water, before administration.

Accordingly, the present invention provides a dispersible tablet with high drug loading comprising Compound I as active ingredient, the active ingredient being present in an amount of from about 42% to 65%, e.g. at least from about 45, 47, 50, 52 or 55% to about 65%, preferably more than 45% by weight based on the total weight of the dispersible tablet. In particular, the amount of Compound I may vary from 42% to 65%, e.g. 45% to 60%, e.g. 45% to 60%, e.g. 45% to 55%, e.g. 47% to 53%, e.g. 50%, by weight based on the total weight of the dispersible tablet.

The present invention pertains to a dispersible tablet comprising an iron-chelating pharmacologically effective amount of Compound I or a pharmaceutically acceptable salt thereof present in an amount of from 42% to 65% by weight based on the total weight of the tablet.

In one aspect of the invention there is provided a dispersible tablet comprising Compound I or a pharmaceutically acceptable salt thereof present in an amount of from about 42% to 65% by weight based on the total weight of the tablet.

Compound I may be in the free acid form or in form of pharmaceutically acceptable salts thereof, preferably in the free acid form. The active moiety corresponds to Compound I in the free acid form. Within the context of this disclosure, reference to Compound I is understood to include Compound I in its free acid form or a pharmaceutically acceptable salt thereof or any crystal forms thereof including hydrates or solvates, if not indicated otherwise and where appropriate and expedient.

The present invention also provides a dispersible tablet comprising:

(a) Compound I or a pharmaceutically acceptable salt thereof, and
(b) at least one pharmaceutically acceptable excipient suitable for the preparation of dispersible tablets wherein the amount of Compound I or a pharmaceutically acceptable salt thereof, calculated as the percentage of the content in weight of the active moiety based on the total weight of the dispersible tablet, is from about 42% to 65%, e.g. at least about 45, 47, 50 or 52%, preferably more than 47% by weight based on the total weight of the dispersible table. In particular, the amount of Compound I as active ingredient may vary from 42% to 65%, e.g. from about 45% to 55%, or from about 47% to 53% by weight based on the total weight of the dispersible tablet.

In a preferred embodiment of the invention, the present invention provides a dispersible tablet wherein Compound I is in the free acid form.

In a most preferred aspect of the invention, Compound I in the free acid form is in a crystalline form.

One or more pharmaceutically acceptable excipients may be present in the dispersible tablets, e.g. those conventionally used, e.g. (1.1) at least one filler, e.g., lactose, ethylcellulose, microcrystalline cellulose, silicified microcrystalline cellulose, e.g. Prosolv™ SMCC®, (1.2) at least one disintegrant, e.g. cross-linked polyvinylpyrrolidinone, e.g. Crospovidone®, (1.3) at least one binder, e.g. polyvinylpyridone, hydroxypropylmethyl cellulose, (1.4) at least one surfactant, e.g. sodium laurylsulfate, (1.5) at least one glidant, e.g. colloidal silicon dioxide, and (1.6) at least one lubricant, e.g. magnesium stearate.

Reference is made to the extensive literature on the subject for these and other pharmaceutically acceptable excipients and procedures mentioned herein, see in particular Handbook of Pharmaceutical Excipients, Third Edition, edited by Arthur H. Kibbe, American Pharmaceutical Association, Washington, USA and Pharmaceutical Press, London; and Lexikon der Hilfsstoffe für Pharmazie, Kosmetik and angrenzende Gebiete edited by H. P. Fiedler, 4th Edition, Edito Cantor, Aulendorf and earlier editions which are incorporated herein by reference.

Fillers (1.1) according to the invention are lactose, especially lactose monohydrate, preferably lactose monohydrate (200 mesh) and lactose spray dried, microcrystalline cellulose, especially PH 102, PH 101 or silicified microcrystalline cellulose, e.g. as known and commercially available under the Trademark Prosolv™ SMCC®90.

Suitable disintegrants (1.2) according to the invention include but are not restricted to maize starch, CMC-Ca, CMC-Na, microcrystalline cellulose, cross-linked PVP, e.g. as known and commercially available under the trade names Crospovidone®, Polyplasdone®, available commercially from the ISP company, or Kollidon® XL, alginic acid, sodium alginate and guar gum. Preferably, cross-linked PVP, e.g. Crospovidone® is used.

Binders (1.3) include but are not restricted to starches, e.g. potato, wheat or corn starch, microcrystalline cellulose, e.g. products such as Avicel®; hydroxypropyl cellulose, hydroxyethyl cellulose, hydroxypropylmethyl cellulose, e.g. hydroxypropylmethyl cellulose-Type 2910 USP, hypromellose, and polyvinylpyrrolidone, e.g. Povidonee®K30 from BASF. Preferably, polyvinylpyrrolidone is used, most preferably PVP K.30.

Appropriate surfactants (1.4) according to the invention may be used: sodium laurylsulfate, quaternary ammonium salts, polysorbates, sorbitan erters and/or poloxamer. Preferably, the surfactant is sodium laurylsulfate.

As glidants (1.5), one or more of the following may be used: silica; colloidal silica, e.g. colloidal silica anhydrous, e.g. Aerosil® 200, magnesium trisilicate, powdered cellulose, starch and talc. Preferably, colloidal silicon dioxide is used.

As lubricants (1.6) one or more of the following may be used Mg-, Al- or Ca-stearate, PEG 4000-8000, talc, sodium benzoate, glyceryl mono fatty acid, e.g. having a molecular weight of from 200 to 800 Daltons, e.g. glyceryl monostearate, e.g. commercially available from Danisco, UK, glyceryl dibehenate, e.g. CompritolATO888™, e.g. commercially available from Gattefossé France), glyceryl palmito-stearic ester, e.g. Precirol™, e.g. commercially available from Gattefossé France, polyoxyethylene glycol, e.g. PEG, e.g. commercially available from BASF, hydrogenated cotton seed oil (Lubitrab, Edward Mendell Co Inc), castor seed oil (Cutina HR, Henkel). Preferably, magnesium stearate is used.

One or more of these pharmaceutically acceptable excipients may be selected and used having regard to the particular desired properties of the dispersible tablet by routine experimentation.

According to the present invention, the amount of fillers (1.1) may vary within a range of from about 30 to 50%, in particular 32 to 48%, e.g. 35 to 45% by weight based on the total weight of the dispersible tablet.

The amount of disintegrant (1.2) may vary within a range of from 2 to 8%, e.g. 4 to 6%, e.g. 5% by weight based on the total weight of the dispersible tablet.

The amount of binder (1.3) may vary from 1 to 10%, preferably from 1.5 to 5% by weight based on the total weight of the dispersible tablet.

The amount of surfactant (1.4) may vary from 0.01 to 3%, preferably from 0.05 to 1.5% by weight based on the total weight of the dispersible tablet.

The amount of glidant (1.5) may vary within ranges of from 0.1 to 5%, in particular 0.1 to 2.5%, e.g. 0.1 to 0.5%, by weight based on the total weight of the dispersible tablet.

The amount of lubricant (1.6) may vary from about 0.45 to 0.85%, e.g. 0.5 to 0.8%, in particular 0.5 to 0.7% in weight based on the total weight of the dispersible tablet.

It will be appreciated that any given excipient may serve more than one function e.g. as filler, disintegrant, binder, glidant, and/or lubricant.

The invention also pertains to a dispersible tablet wherein the lubricant is magnesium stearate.

In a preferred aspect of the invention, the dispersible tablet comprises the following pharmaceutically acceptable excipients: one or more fillers in a total amount of about 30% to 50% by weight based on the total weight of the dispersible tablet, one or more binders in a total amount of about 1.5% to 5% by weight based on the total weight of the dispersible tablet, one or more disintegrants in a total amount of about 2% to 8% by weight based on the total weight of the dispersible tablet, one or more surfactant in a total amount of about 0.01% to 1.5% by weight based on the total weight of the dispersible tablet, one or more glidants in a total amount of about 0.1% to 5% by weight based on the total weight of the dispersible tablet, and one or more lubricants in a total amount of about 0.45% to 0.85% by weight based on the total weight of the dispersible tablet.

In a preferred aspect of the invention, the dispersible tablet comprises the following pharmaceutically acceptable excipients: one or more fillers in a total amount of about 35% to 45% by weight based on the total weight of the dispersible tablet, one or more binders in a total amount of about 1.5% to 5% by weight based on the total weight of the dispersible tablet, one or more disintegrants in a total amount of about 2% to 8% by weight based on the total weight of the dispersible tablet, one or more surfactant in a total amount of about 0.01% to 1.5% by weight based on the total weight of the dispersible tablet, one or more glidants in a total amount of about 0.1% to 2.5% by weight based on the total weight of the dispersible tablet, and/or one or more lubricants in a total amount of 0.45% to 0.85% by weight based on the total weight of the dispersible tablet.

The absolute amounts of each pharmaceutically acceptable excipient and the amounts relative to other pharmaceutically acceptable excipients is similarly dependent on the desired properties of the dispersible tablet and may also be chosen by routine experimentation.

The present inventors have encountered difficulties in the production of dispersible tablets comprising Compound I which may be due to the low density of the active ingredient, to its electrostatic characteristics which may lead to a poor flowability and to its sticking tendency.

In accordance with the present invention, it has now unexpectedly been found that pharmaceutically acceptable oral solid dosage forms in the form of dispersible tablets convenient for patient administration and dispersible in 5 minutes or less, preferably 3 minutes or less, may be obtained by preparation of tablets by compression methods. More specifically, the dispersible tablets of the invention may be prepared by granulation, preferably wet-granulation, followed by compression methods, and conventional lubrication process or alternatively under spray lubrication.

The present inventors have encountered difficulties in the production of dispersible tablets comprising Compound I in an amount of 1000 mg using the method described in WO 2004/035026 because it leads to a tablet of more than 3000 mg having the following drawbacks. They are friable to handle, pack and transport without damaging the tablet. The inventors have now surprisingly found that it is possible to granulate Compound I without adding excipients other than the binder and the surfactant, e.g. present in the granulating solution, leading the granulate solely composed of Compound I, one or more binder, e.g. PVP K30 and one or more surfactant, e.g. sodium lauryl sulfate. This method allows to increase the drug load of the granulate and to manufacture a dispersible tablet, having a total weight of 2000 mg±5% comprising Compound I in an amount of 1000 mg±5%.

In general, wet-granulation may be used to improve flowability and sticking tendency, however, wet-granulation process is not preferred when the pharmaceutical composition is to be a dispersible tablet. Wet-granulation increases the cohesion of the active ingredient particles and increases the disintegration time of the final tablet which is not in accordance with patient compliance or the European Pharmacopoeia which requests a disintegration time of 3 minutes or less for a dispersible tablet.

The dispersible tablets of the invention have a disintegration time, e.g. in aqueous media, e.g. in water, of 5 minutes or below 5 minutes. The dispersible tablets of the invention are, despite the high drug loading, dispersible, e.g. in aqueous media, e.g. in water, in less than 5 minutes, preferably less than 3 minutes, and, therefore, convenient to administer, e.g. to patients. This leads to a better patient compliance.

In another embodiment, this invention provides a dispersible tablet comprising more than 800 mg of Compound I as active ingredient, e.g. of from 900 mg to about 1100 mg, e.g. 1000 mg. Most preferably, dispersible tablets according to the invention are dispersible tablets containing 1000 mg of Compound I as active ingredient.

Accordingly, the present invention provides dispersible tablets, e.g. dispersible tablets, containing an amount of Compound I, equal to 1000 mg of Compound I free acid form. Most preferably, the Compound I in the free acid form used for the dispersible tablet according to the invention is the crystalline form, especially the crystalline form the preparation of which is described in example 5 of WO 97/49395, which is hereby incorporated by reference.

According to the invention, the process for the preparation of the dispersible tablets consists of granulating an inner phase, mixing (together) it with one or more pharmaceutically acceptable excipient(s) and compressing the obtained mixture, optionally under spray lubrication conditions.

The inner phase comprises Compound I. The inner phase is granulated with the granulation liquid. The granulation liquid may be an aqueous solution, e.g. comprising one or more surfactant and/or one or more binder, e.g. an aqueous solution of sodium laurylsulfate and PVP K.30. The Compound I is mixed with a wetting solution comprising one or more surfactants, water and one or more binders. The preferred binder is PVP K.30. The mixture is processed for granulation, e.g. using a wet high-shear granulator to form the wet-granulates. The wet-granulates may then be dried, e.g. using a fluid bed dryer, and calibrated, e.g. using an oscillating granulator.

The outer phase consists of one or more pharmaceutically acceptable excipient(s) and is mixed with the inner phase using, e.g. a free fall mixer. Preferably, one or more fillers and one or more glidants are added and/or one or more disintegrant and/or one or more lubricant. Preferably, cellulose microcrystalline, and/or lactose, e.g. lactose monohydrate, lactose spray dried, are added as fillers, most preferably the fillers are cellulose microcrystalline and lactose, e.g. lactose spray dried. Preferably the amount of one or more fillers in the outer phase is ranging from about 5 to 50% by weight based on the total weight of the dispersible tablet, more preferably from about 10 to 45%. Even more preferably, microcrystalline cellulose is added in the range of 5 to 20% in weight based on the total weight of the dispersible tablet. Lactose, e.g. lactose spray dried, is added in the range of 15 to 35% by weight based on the total weight of the dispersible tablet. The disintegrant is preferably Crospovidone XL. The amount of disintegrant present in the inner phase is preferably ranging from 2 to 8%, preferably 4 to 6%, e.g. about 5% by weight based on the total weight of the dispersible tablet. The outer phase according to the invention may also contain one or more glidants, most preferably colloidal silicon dioxide. In one embodiment, the amount of glidant in the outer phase is ranging from about 0.1 to 5%, preferably from about 0.1 to 2.5%, most preferably from about 0.1 to 1%, e.g. 0.5%, in weight based on the total weight of the tablet. The outer phase according to the invention may also contain one or more lubricant in an amount of from about 0.45 to 0.85%, preferably 0.5 to 0.8%, e.g. 0.5 to 0.7%, e.g. 0.5% in weight based on the total weight of the tablet.

Optionally, according to the present invention, one or more lubricants, in addition of being incorporated into the mixture of the inner and outer phase, may be deposited on the punches of the tabletting machine before compression. According to the invention, one or more lubricants may be sprayed on the material contacting surfaces of pressing tools, e.g. punches and/or dies, of the tabletting machine before compression. Preferably, one or more lubricants are sprayed on the material contacting surfaces of pressing tools, e.g. punches and dies, of the tabletting machine before compression.

In one embodiment of the invention, the process for the preparation of a dispersible tablet comprises

(a) wet-granulating an inner phase comprising
(i) Compound I or a pharmaceutically acceptable salt thereof
   (b) forming an outer phase comprising
(ii) adding pharmaceutically acceptable excipients to the inner phase obtained in
(i) and mixing;
   (c) lubricating the mixture obtained in step (ii)
(iii) by adding one or more lubricants to the blend obtained in (ii) and mixing;
   (d) forming the dispersible tablet by
(iv) compressing the mixture obtained in step (iii), optionally under spray lubrication condition.

In a further aspect, the present invention provides a process comprising:

(i) Compound I or a pharmaceutically acceptable salt thereof;
(ii) adding a solution comprising one or more surfactant(s) and one or more binder(s), subjecting the mixture to wetting/kneading, e.g. in a high shear mixer, wet-granulating using, e.g. a rotating impeller, drying, e.g. in a fluidized bed dryer, then calibrating in an oscillating granulator, and;
(iii) adding pharmaceutically acceptable excipients, e.g. sieved excipients, such as one or more fillers, e.g. microcrystalline cellulose or lactose, e.g. lactose spray dried, one or more disintegrant, e.g. CrospovidoneXL, and one or more glidant, e.g. colloidal silicon dioxide, and mixing, e.g. in a free fall mixer;
(iv) adding one or more lubricant, e.g. magnesium stearate, and mixing, e.g. in a free fall mixer;
(v) tabletting the mixture obtained in step (iii) by compression, e.g. in a conventional tablet press, preferably a rotary machine, and
(vi) optionally, spraying the lubricant on the materials contacting surfaces of pressing tools.

Procedures which may be used may be conventional or known in the art or based on such procedures e.g. those described in L. Lachman et al. The Theory and Practice of Industrial Pharmacy, 3rd Ed, 1986, H. Sucker et al, Pharmazeutische Technologie, Thieme, 1991, Hagers Handbuch der pharmazeutischen Praxis, 4th Ed. (Springer Verlag, 1971) and Remington's Pharmaceutical Sciences, 13th Ed., (Mack Publ., Co., 1970) or later editions.

By “inner phase” is meant the granulate phase (steps (i) and (ii)) including the active ingredient Compound I and optionally one or more pharmaceutically acceptable excipients.

By “outer phase” is meant one or more pharmaceutically acceptable excipients added to the inner phase (granulates).

By “total weight of the dispersible tablet” is meant the weight of a tablet being the inner and the outer phase.

The physical and chemical stability may be tested in a conventional manner, e.g. the dispersible tablets may be tested as such by measurement of dissolution, friability, disintegration time, assay for Compound I degradation products, appearance and/or microscopy, e.g. after storage at room temperature, i.e. 25° C., and/or storage at 40° C.

The dispersible tablets may vary in shape and be, for example, round, oval, oblong, cylindrical or any other suitable shape. In a preferred embodiment of the invention dispersible tablets obtained by the compression method described above are of elongated shape. The edges of the dispersible tablets may be beveled or rounded. Most preferably, the dispersible tablets are of elongated shape with beveled edges. The dispersible tablets according to the invention may be scored, embossed or engraved.

The dispersible tablet according to the invention is preferably of elongated shape, flat, optionally with beveled edges. The dispersible tablet comprising 1000 mg of Compound I as active ingredient has a size ranging from 20 to 26 mm for length and 10 to 18 mm for width, preferably said dispersible tablet has a length of 24 mm and a width of 12 mm. The 1000 mg tablet has a thickness ranging from 5 to 10 mm, preferably 5.5 to 8.5 mm.

The dispersible tablets of the invention comprising about 1000 mg of Compound I as active moiety may have a hardness of from about 100 to 220 N, e.g. 120 to 200 N, preferably 140 to 180 N, e.g. as measured with a conventional hardness tester.

Preferably, the disintegration time is not more than 5 minutes, most preferably the disintegration time is less than 3 minutes as measured using a disintegration time apparatus.

By “disintegration time” is meant the time that needs the dispersible tablet to disintegrate in water at room temperature in a disintegration time device.

The dispersible tablet of the present invention is dispersible in an aqueous phase, preferably water.

The dispersible tablets of the invention may be colored and/or marked so as to impart an individual appearance and to make them instantly recognizable. The use of dyes can serve to enhance the appearance as well as to identify the dispersible tablets. Dyes suitable for use in pharmacy typically include carotinoids, iron oxides or chlorophyll. The dispersible tablets of the invention may be marked using an imprint code.

The dispersible tablets of the invention are useful for the treatment of iron overload in transfusion dependent anemias, in particular thalassemia major, thalassemia intermediate and sickle cell disease and in the treatment of hemochromatosis.

The activity and characteristics of the dispersible tablets of the invention may be indicated in standard clinical trials and/or animal trials.

The dispersible tablets of the invention are stable during storage, e.g. for 2 years or even 3 years in conventional packaging, e.g. blister packs. Less than about 5%, e.g. 2 or 3%, or less of Compound I as active ingredient may degrade during this time as determined in conventional tests.

The invention further relates also to a method of administering to a mammal, preferably a human subject, in need of such a treatment, Compound I or a pharmaceutically acceptable salt thereof in the form of a dispersible tablet. The invention relates especially to such method wherein a daily dose of 5 to 40 mg/kg of body weight of Compound I as active ingredient is administered to a patient. It will be understood that the specific dose level for any particular patient will depend upon a variety of factors including the age, the body weight, general health, drug combination with one or more active drugs, type and severity of the disease.

The invention further provides a medicament package comprising dispersible tablets according to the invention and printed instructions directing that one or more dispersible tablets of Compound I or a pharmaceutically acceptable salt thereof be administered orally.

The following non-limitative examples illustrate the invention.

### EXAMPLE 1

| | | | Quantity |
|---|---|---|---|
| | | Quantity per unit | per |
| Process step | Component | [mg] | batch [%] |
| Inner Phase | Micronized Compound I | 1000.0 | 50.0 |
| | Polyvinylpyrrolidon K30 | 60.0 | 3.0 |
| | Sodium laurylsulfate | 2.0 | 0.1 |
| Outer Phase | Crospovidone XL | 100.0 | 5.0 |
| | Microcrystalline cellulose | 200.0 | 10.0 |
| | Lactose spray-dried | 618.0 | 30.9 |
| | Colloidal Silicon dioxide | 10.0 | 0.5 |
| | Magnesium stearate | 10.0 | 0.5 |
| Total | | 2000.0 | 100.0 |

### EXAMPLE 2

### Example of Disintegration Times

The following table provides examples of disintegration times in minutes of tablets formulated according to Example 1.

| | Laboratory phase | Pilot phase |
|---|---|---|
| Time point | (Batch size: ca. 24 kg) | (Batch size: ca. 200 kg) |
| 1 | 1.73 | 2.08 |
| 2 | 1.68 | 2.24 |
| 3 | 1.60 | 2.38 |
| 4 | 1.65 | 2.11 |

1: A dispersible tablet comprising (a) Compound I of the formula 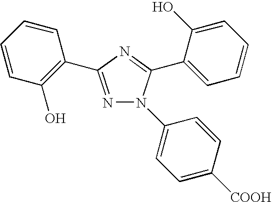
or a pharmaceutically acceptable salt thereof present in an amount of from 42% to 65% by weight based on the total weight of the tablet and (b) at least one pharmaceutically acceptable excipient suitable for the preparation of dispersible tablets wherein said pharmaceutically acceptable excipients are:
(i) at least one filler in a total amount of about 35 to 45% by weight based on the total weight of the tablet,
(ii) at least one disintegrant in a total amount of about 2% to 8% by weight based on the total weight of the tablet
(iii) at least one binder in a total amount of about 1% to 5% by weight based on the total weight of the tablet,
(iv) at least one surfactant in a total amount of about 0.01% to 1% by weight based on the total weight of the tablet,
(v) at least one glidant in a total amount of about 0.1% to 5% by weight based on the total weight of the tablet and
(vi) at least one lubricant present in a total amount of about 0.45 to 0.85% by weight based on the total weight of the tablet

2: The dispersible tablet according to claim 1 wherein Compound I is in the free acid form.

3: The dispersible tablet according to claim 1 wherein Compound I is in a crystalline form.

4: The dispersible tablet according to claim 1 wherein the disintegration time of the tablet is of 5 minutes or less.

5: The dispersible tablet according to claim 4 wherein the disintegration time of the tablet is of 3 minutes or less.

6: The dispersible tablet according to claim 1 wherein the lubricant is magnesium stearate.

7: The dispersible tablet according to claim 1 containing Compound I in its free acid form in an amount of about 900 mg to 1100 mg.

8: A process for the preparation of the dispersible tablet according to claim 1, which process comprises
(a) wet-granulating an inner phase comprising
(i) Compound I or a pharmaceutically acceptable salt thereof;
(b) forming an outer phase comprising
(ii) adding further pharmaceutically acceptable excipients to the inner phase obtained in (i) and mixing;
(c) lubricating the mixture obtained in step (ii)
(iii) by adding one or more lubricants to the blend obtained in (ii) and mixing;
(d) forming the dispersible tablet by
(iv) compressing the mixture obtained in step (iii), optionally under spray lubrication condition.

9: A medicament package comprising the dispersible tablets according to claim 1 together with instructions for administration.

10: A dispersible tablet obtainable by the process of claim 8.

11. (canceled)

12: A method of treating a patient suffering from iron overload in transfusion dependent anemias comprising administering the dispersible tablet according to claim 1.
